(19) United States
(12) Patent Application Publication
(10) Pub. No.: US 2025/0369990 A1
(43) Pub. Date: Dec. 4, 2025

(54) THC ENZYME SENSOR

(71) Applicant: Consumer Safety Technology, LLC, Des Moines, IA (US)

(72) Inventor: Evan Rashied Darzi, Phoenix, AZ (US)

(21) Appl. No.: 19/210,505

(22) Filed: May 16, 2025

Related U.S. Application Data
(60) Provisional application No. 63/654,855, filed on May 31, 2024.

(51) Int. Cl. G01N 33/94; A61B 5/08; A61B 5/097; G01N 33/543

(52) U.S. Cl. CPC G01N 33/948; A61B 5/082; A61B 5/097; G01N 33/5438

(57) ABSTRACT

Embodiments herein relate to THC detection devices. A method of detecting THC compounds, is included, the method includes receiving an exhaled breath sample; heating a capture structure to a temperature to vaporize THC compounds; oxidizing the vaporized THC compounds on a detector element, wherein the detector element includes a THC targeted enzyme substrate; and detecting an amount of the THC compounds present in the exhaled breath sample. Other embodiments are also included herein.

This application claims the benefit of U.S. Provisional Application No. 63/654,855, filed May 31, 2024, the content of which is incorporated herein by reference in its entirety.

### FIELD

Embodiments herein relate generally to THC detection devices, and more specifically the use of THC as a substrate in an enzyme catalyzed redox reaction in THC detection devices.

### BACKGROUND

Breath alcohol detection devices are used to measure an amount of alcohol in a user's breath. It is known that concentration of alcohol in a user's breath is closely proportional to the concentration of alcohol in the user's blood, which is typically the basis upon which intoxication is legally determined. Generally, a user blows into a mouthpiece of an alcohol detection device and a breath path is configured to transport at least a portion of the breath sample to a sensing element of the detection device. The capability to detect an amount of phenolic cannabinoid, such as tetrahydrocannabinol (“THC”), in a user's breath, would be valuable for law enforcement, employers, and accountability partners. The concentration of phenolic cannabinoid in a user's breath typically correlates with recent use of cannabinoid products, such as marijuana.

### BRIEF DESCRIPTION OF THE FIGURES

Aspects may be more completely understood in connection with the following figures (FIGS.), in which:

FIG. 1 is a schematic view of a detection device in accordance with various embodiments herein.

FIG. 2 is a schematic view of a detector element in accordance with various embodiments herein.

FIG. 3 is a THC oxidation reaction in accordance with various embodiments herein.

FIG. 4 is a method in accordance with various embodiments herein.

FIG. 5 is a method in accordance with various embodiments herein.

FIG. 6 is a method in accordance with various embodiments herein.

FIG. 7 is a computerized detection system in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

The ability to measure the amount of THC in a user's breath, or other sample, can be valuable for law enforcement, employers, and accountability partners. However, THC in the breath is often present in very low concentrations and may include contaminants. This can lead to complications in both selectivity and sensitivity. Previous attempts to overcome these problems have relied on chemical adsorption techniques to capture and concentrate the THC using polar silica gel or non-polar C18 silica and requiring solvent to elute the sample in a laboratory setting. This approach involves single use disposable cartridges, expensive laboratory protocols, and an inability to provide forensic results outside of a laboratory setting.

Embodiments herein overcome these complications using enzymes which can accept THC as a substrate for redox chemistry and thus overcome the selectivity and sensitivity complications. This approach offers the possibility to not require the use of disposable cartridges or laboratory testing.

Embodiments herein relate to THC detection devices using THC targeted enzymes. In various embodiments, the THC targeted enzymes can oxidize or reduce THC in a sample matrix. The THC detection device can measure the amount of THC present in the sample based on the amount of oxidized THC measured.

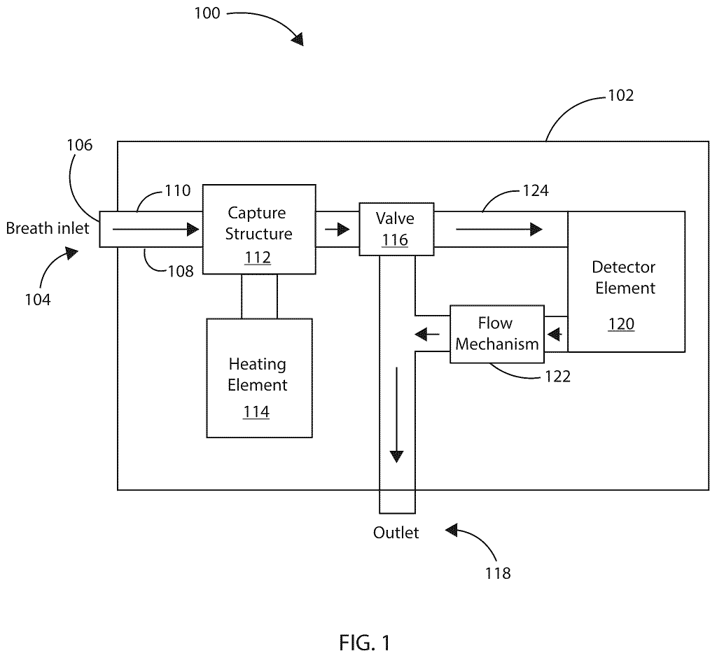

### Detection Device (FIG. 1)

Referring now to FIG. 1, a schematic view of a THC detection device is shown in accordance with various embodiments herein. In various examples, the detection device can detect a substance such as cannabis in a sample, such as a breath sample. The detection device 100 can include a housing 102 and a breath inlet 104. The housing 102 is preferably a relatively hard durable material that serves to protect the internal components of the detection device 100. The breath inlet 104 can be positioned on a side of the housing 102.

Detection device 100 can be used to measure an amount of phenolic cannabinoid, such as tetrahydrocannabinol, in a user's breath. The concentration of phenolic cannabinoid in a user's breath typically correlates with recent use of cannabinoid products, such as marijuana. Generally, a user blows into a mouthpiece of a phenolic cannabinoid detection device, and a breath path is configured to transport at least a portion of the breath sample to a detector element of the detection device.

### Breath Opening/Mouthpiece

The breath inlet 104 can define a breath inflow opening 106. The breath inflow opening 106 can be configured to receive a user's breath. The breath inlet 104 can receive the mouth of the user providing a breath sample to the detection device 100. The breath inlet 104 can be configured to facilitate the user's mouth sealing against an exterior surface of the breath inlet 104. Alternatively, the breath inlet 104 can be configured to receive a breath sample that is provided where the user is spaced apart from the breath inlet 104 and is directing breath toward the breath inlet 104 from a distance.

In various embodiments, the breath inlet 104 can be configured to be removably attachable to the detection device 100. In some embodiments, the breath inlet 104 can include a mouthpiece. The mouthpiece can be removable by means of a friction or snap fit, or similar mechanism. This permits each user to have a separate mouthpiece for sanitary reasons, it also permits easy cleaning or replacement of the mouthpiece. In various embodiments, the breath inlet 104 can be formed from a substantially rigid material configured to retain its shape when a breath sample is provided to the detection device 100. Alternatively, the breath inlet 104 can be formed from a compliant material configured to conform to a user's mouth when a breath sample is provided to the detection device 100. The breath inlet 104 can be made from any suitable material or materials including but not limited to plastics, rubbers, silicone, metals, or the like.

In various embodiments, the user's breath can travel into the breath inflow opening 106 and through a breath conduit path 108. The breath conduit path 108 can define a breath path 110. In some embodiments, the breath conduit path 108 is connected to a capture structure 112 discussed below. In other embodiments, the breath conduit path 108 is connected to a heating element 114, discussed below. The user's breath can travel into the breath inflow opening 106, through the breath path 110, and into the capture structure 112. It is herein contemplated that the capture structure 112 can capture one or more breaths of the user. In various embodiments, the capture structure 112 can capture one, two, three, four, five, six, seven, eight, nine, or ten breaths. For example, the capture structure 112 can capture one, two, three, four, or five breaths of the user.

### Capture Structure

In various embodiments, the capture structure 112 can include a material designed to capture or trap components found in the sample, such as the user's breath.

### Compounds of Interest

Components of a sample can include compounds of interest which the detector element is designed to detect, such as cannabis. It is herein contemplated that cannabis, including a variety of cannabis metabolites or compounds, can be compounds of interest. Cannabis metabolites and cannabis compounds can include, but are not limited to, cannabinoids, phenolic cannabinoids, Δ⁹-tetrahydrocannabinol (Δ⁹-THC), Δ⁸-tetrahydrocannabinol (Δ⁸-THC), cannabinol (CBN), cannabidiol (CBD), 11-hydroxy-49-THC (11-OH-THC), anandamide (arachidonylethanolamide), cannabichromene, and (−)Δ⁸-THC-11-oic acid).

Components of the sample can include contaminants such as alcohol, glucose, ethanol, acetone, nitric oxide, carbon monoxide, isoprene, ethane, pentane, water, and the like. It is noted that contaminants can also be considered compounds of interest.

### Heating Element

In various embodiments, after components in the user's breath are deposited on the capture structure 112, a heating element 114 can provide heat to the capture structure 112. The heating element 114 can be configured to increase the temperature of the capture structure 112 from a starting temperature, such as room temperature to one or more desired temperatures. In some embodiments, the desired temperature can be a temperature sufficient to vaporize one or more components of the breath sample. In some embodiments, the desired temperature can be at least the boiling point of one or more components in the breath sample. For example, the desired temperature could be at least 157° C., the boiling point of cannabis, or at least 170° C.

### Valve

In various embodiments, the detection device 100 can include a valve 116. It is herein contemplated that the valve 116 can be a variety of different valves. For example, the valve 116 can include a solenoid valve, a butterfly valve, a diaphragm valve, a gauge valve, a check valve, and the like.

In various embodiments, the valve 116 can be configured to direct the vaporized components coming off the capture structure 112. In a first position, the valve 116 can connect the capture structure 112 with the outlet 118, so that vaporized contaminants, such as water and ethanol, can be drawn out of the detection device 100. In a second position, the valve 116 can connect the capture structure 112 with the detector element 120, so that vaporized components of interest, such as cannabis can be drawn into the detector element 120. In an optionally third position, the valve 116 can close off vapors coming from the capture structure 112 from an outlet 118 and a detector element 120.

### Flow Mechanism

In various embodiments, the vaporized components of interest can be drawn into the detector element 120 via a flow mechanism 122, such as a pump. For example, the pump can provide a vacuum or negative pressure through tube 124 and draw the vaporized components through the detector element 120.

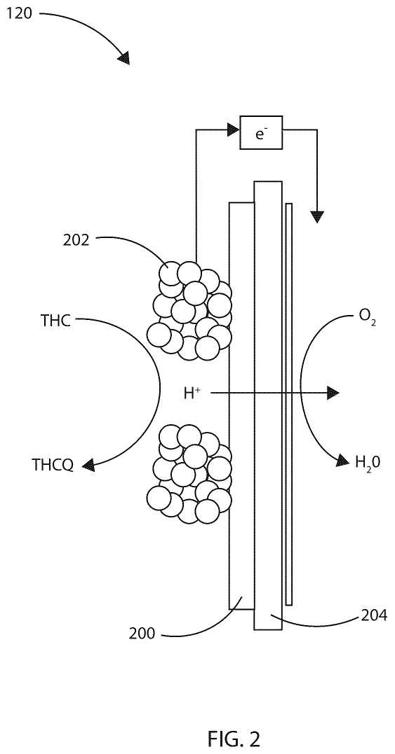

### Detector Element (FIG. 2)

Referring now to FIG. 2, a schematic view of a detector element is shown in accordance with various embodiments herein. In various embodiments, the detector element 120 can include a detector configured to measure vaporized components of interest in a sample. For example, the detector element 120 can be configured to measure the amount of cannabis in the user's breath. The detector element 120 can include, but is not limited to, a variety of sensors such as electrochemical sensors, fuel cells, chemiresistors, and voltametrics.

In various embodiments, the detector element can include a THC targeted enzyme substrate 200. The THC targeted enzyme can be provided by any developer of enzymes. This enzyme could be arrived at through exhaustive screening of known redox active enzymes or directed evolution of known enzymes to arrive at a novel enzyme for THC redox chemistry. In various embodiments, vaporized components, including THC molecules 202, can be drawn into the detector element 120 and land on the THC targeted enzyme substrate 200. The detector element 120 can then measure the amount of THC in the user's breath, explained in greater detail below.

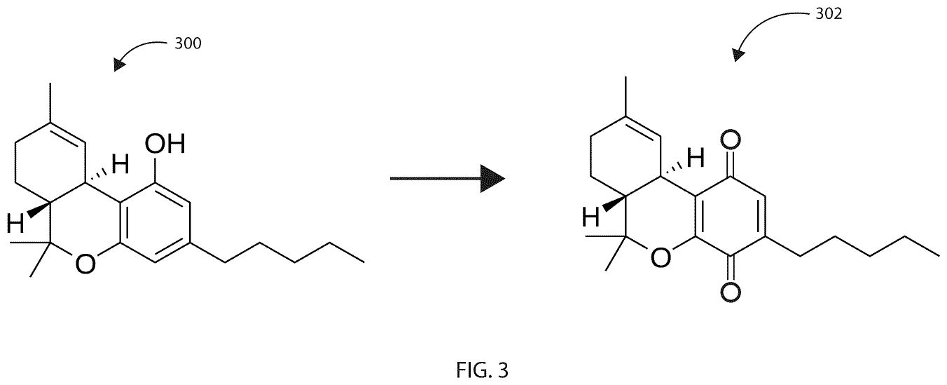

### THC Oxidation (FIGS. 2 and 3)

In various embodiments, once the THC makes contact with or lands on surface 204 of the THC targeted enzyme substrate 200, the THC molecules 202 can begin to undergo an enzymatic oxidation reaction. During the oxidation, electrons from the THC molecules 202 are released and transferred to the surface 204 of the detector element 120. An electric current is generated by the transfer of electrons from the THC molecules 202 to the detector element 120 which can be measured. It is noted that the current generated is proportional to the rate of the oxidation reaction which is directly proportional to the concentration of THC in the sample.

The oxidation reaction of THC is shown in FIG. 3. Referring now to FIG. 3, a THC oxidation reaction is shown in accordance with various embodiments herein. In various embodiments, a THC targeted enzyme can catalyze the oxidation of THC molecule 300 to produce oxidized THC 302.

### THC Reduction

In alternative embodiments, the THC molecules 202 can undergo a THC reduction reaction once the THC makes contact with or lands on the surface 204 of the THC targeted enzyme substrate 200. During the reduction, electrons from the THC molecules 202 can be absorbed and transferred from the surface 204 of the detector element 120. An electric current is generated by the transfer of electrons to the THC molecule 202 to the detector element 120 which can be measured. Similar to the oxidation reaction, it is noted that the current generated is proportional to the rate of the reduction reaction which is directly proportional to the concentration of THC in the sample.

### Sample and Compounds of Interest

Throughout the application, breath is described as a sample that is analyzed for the presence of a substance such as an intoxicant. It is also possible for the embodiments of the application to be used to process a sample different than breath, such as another gas sample, such as environmental or ambient air or vapor from skin, or another biological sample, such as saliva, mucous, or urine.

Throughout the application, cannabis is described as a substance of interest or compounds of interest that is detected by a detector element. It is also possible for other substances and compounds to be detected by a detector element in the various embodiments described herein, such as different intoxicants, prescription drugs, cocaine, heroin, nicotine, methamphetamine, amphetamines, hallucinogens, or other substances. It is noted that each intoxicant would be oxidized using an intoxicant-specific enzyme.

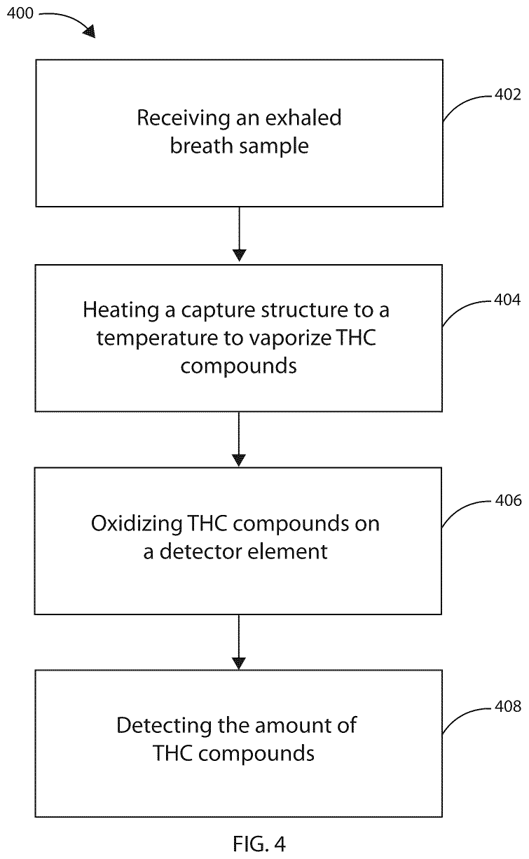

### Methods (FIG. 4-6)

Many different methods for THC detection using a THC targeted enzyme are contemplated herein, including, but not limited to, methods of making, methods of using, and the like. Aspects of system/device operations described elsewhere herein can be performed as operations of one or more methods in accordance with various embodiments herein.

Referring now to FIG. 4, a flow diagram of a method is shown in accordance with various embodiments herein. FIG. 4 shows a method 400 of detecting THC compounds. The method can include receiving an exhaled breath sample 402.

The method can further include heating a capture structure to a temperature to vaporize THC molecules 404. It is noted herein that the capture structure can first be heated to a temperature below the boiling point of THC. In various embodiments, heating below the boiling point of THC can allow for volatile contaminants, such as ethanol or water found in the breath sample to be removed from the capture structure. In various embodiments, the volatile contaminants are exhausted out of the detector device. It is noted that exhausting the volatile contaminants out of the detector device can be beneficial to preventing the contaminants from reaching the detector element. By preventing the contaminants from reaching the detector element, the detector element can remain free of contaminants while also providing accurate measurements of THC.

The method can further include oxidizing THC compounds on a detector element 406. In various embodiments, the THC compounds can be oxidized by coming into contact with a THC targeted enzyme substrate positioned on a surface of the detector element.

The method can further include detecting the amount of THC compounds 408. It is noted that by oxidizing the THC compounds, an electrical current is produced that can be measured by the detector element. Further, the current generated is directly proportional to the amount of THC present in the user's breath.

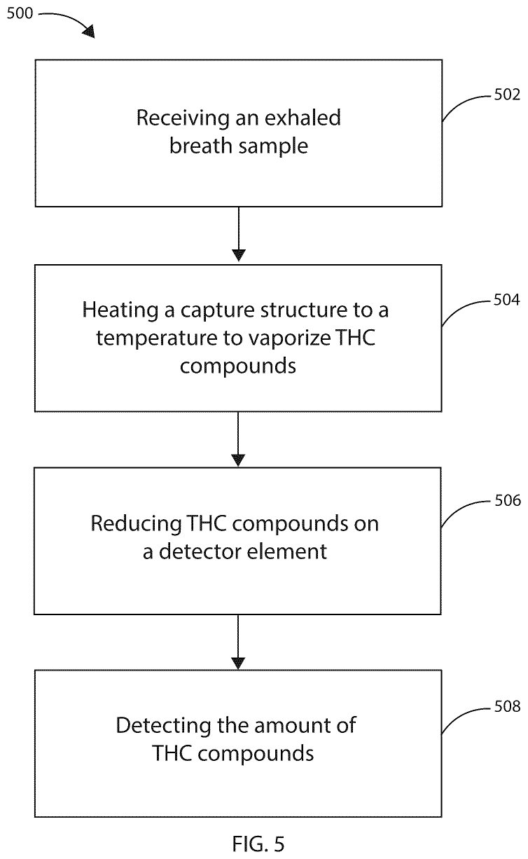

Referring now to FIG. 5, a flow diagram of a method is shown in accordance with various embodiments herein. FIG. 5 shows a method 500 of detecting THC compounds. The method can include receiving an exhaled breath sample 502.

The method can further include heating a capture structure to a temperature to vaporize THC molecules 504. As discussed above with respect to FIG. 4, the capture structure can first be heated to a temperature below the boiling point of THC. In various embodiments, heating below the boiling point of THC can allow for volatile contaminants, such as ethanol or water found in the breath sample to be removed from the capture structure. In various embodiments, the volatile contaminants are exhausted out of the detector device. It is noted that exhausting the volatile contaminants out of the detector device can be beneficial to preventing the contaminants from reaching the detector element. By preventing the contaminants from reaching the detector element, the detector element can remain free of contaminants while also providing accurate measurements of THC.

The method can further include reducing THC compounds on a detector element 506. In various embodiments, the THC compounds can be reduced by coming into contact with a THC targeted enzyme substrate positioned on a surface of the detector element.

The method can further include detecting the amount of THC compounds 508. It is noted that by reducing the THC compounds, an electrical current is produced that can be measured by the detector element. Further, the current generated is directly proportional to the amount of THC present in the user's breath.

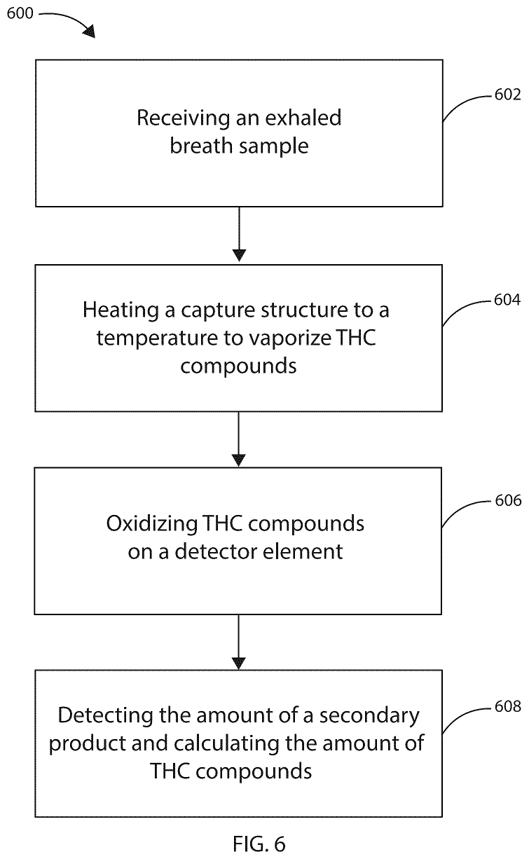

In various embodiments, the amount of THC compounds present in a breath sample can be calculated by detecting an amount of one or more secondary products present in the oxidation or reduction reaction. Referring now to FIG. 6, a flow diagram of a method is shown in accordance with various embodiments herein. FIG. 6 shows a method 600 of detecting THC compounds. The method can include receiving an exhaled breath sample 602.

The method can further include heating a capture structure to a temperature to vaporize THC molecules 604. The capture structure can first be heated to a temperature below the boiling point of THC. In various embodiments, heating below the boiling point of THC can allow for volatile contaminants, such as ethanol or water found in the breath sample to be removed from the capture structure. In various embodiments, the volatile contaminants are exhausted out of the detector device. It is noted that exhausting the volatile contaminants out of the detector device can be beneficial to preventing the contaminants from reaching the detector element. By preventing the contaminants from reaching the detector element, the detector element can remain free of contaminants while also providing accurate measurements of THC.

The method can further include oxidizing THC compounds on a detector element 606. In various embodiments, the THC compounds can be oxidized by coming into contact with a THC targeted enzyme substrate positioned on a surface of the detector element. In other embodiments, the THC compounds can be reduced on the detector element. It is noted that when THC is either oxidized or reduced on the detector element, one or more secondary products, such as hydrogen peroxide are produced.

The method can further include detecting the amount of a secondary product and calculating the amount of THC compounds 608. In various embodiments, the detector element can be configured to measure the amount of electrical current produced by the secondary product. The electrical current generated by the amount of secondary product is directly proportional to the amount of THC present in the user's breath. As such, the amount of THC present in a breath sample can be indirectly calculated by directly measuring the amount of secondary product measured on the detector element.

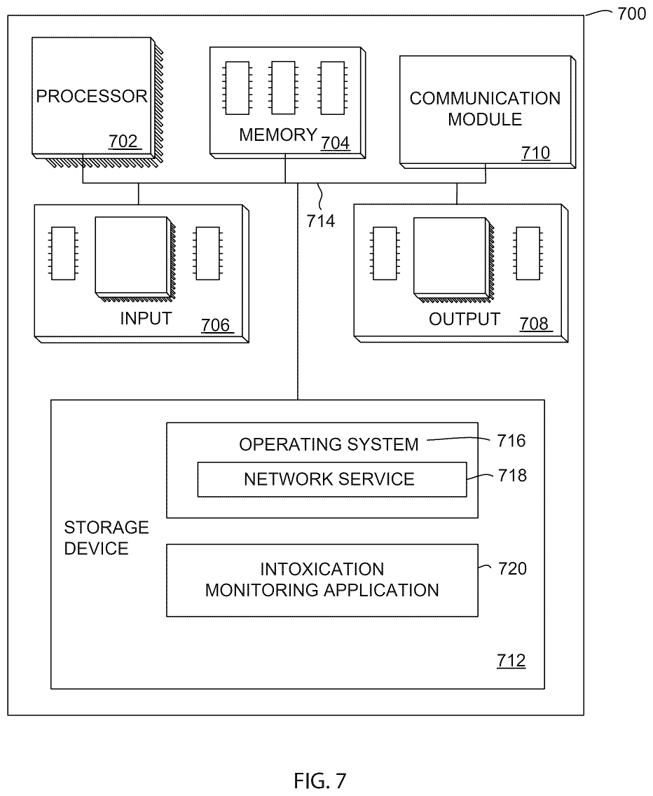

### Computer Systems (FIG. 7)

The systems and methods presented here may be implemented in part using a computerized device, such as a smartphone, handheld, or other computerized device. FIG. 7 shows a computerized detection system consistent with various examples described herein. FIG. 7 illustrates only one particular example of computing device 700, and other computing devices 700 may be used in other embodiments. Although computing device 700 is shown as a standalone computing device, computing device 700 may be any component or system that includes one or more processors or another suitable computing environment for executing software instructions in other examples and need not include all the elements shown here. As shown in the specific example of FIG. 7, computing device 700 includes one or more processors 702, memory 704, one or more input devices 706, one or more output devices 708, one or more communication modules 710, and one or more storage devices 712. Computing device 700, in one example, further includes an operating system 716 executable by computing device 700. The operating system includes in various examples services such as a network service 718. One or more applications, such as a breath intoxicant detection application 720, are also stored on storage device 712 and are executable by computing device 700.

Each of components 702, 704, 706, 708, 710, and 712 may be interconnected (physically, communicatively, and/or operatively) for inter-component communications, such as via one or more communication channels 714. In some examples, communication channels 714 include a system bus, network connection, inter-processor communication network, or any other channel for communicating data. Applications such as breath intoxicant detection application 720 and operating system 716 may also communicate information with one another as well as with other components in computing device 700.

Processors 702, in one example, are configured to implement functionality and/or process instructions for execution within computing device 700. For example, processors 702 may be capable of processing instructions stored in storage device 712 or memory 704. Examples of processors 702 include any one or more of a microprocessor, a controller, a digital signal processor (DSP), an application specific integrated circuit (ASIC), a field-programmable gate array (FPGA), or similar discrete or integrated logic circuitry.

One or more storage devices 712 may be configured to store information within computing device 700 during operation. Storage device 712, in some examples, is known as a computer-readable storage medium. In some examples, storage device 712 comprises temporary memory, meaning that a primary purpose of storage device 712 is not long-term storage. Storage device 712 in some examples includes a volatile memory, meaning that storage device 712 does not maintain stored contents when computing device 700 is turned off. In other examples, data is loaded from storage device 712 into memory 704 during operation. Examples of volatile memories include random access memories (RAM), dynamic random-access memories (DRAM), static random-access memories (SRAM), and other forms of volatile memories known in the art. In some examples, storage device 712 is used to store program instructions for execution by processors 702. Storage device 712 and memory 704, in various examples, are used by software or applications running on computing device 700 such as breath intoxicant detection application 720 to temporarily store information during program execution.

Storage device 712, in some examples, includes one or more computer-readable storage media that may be configured to store larger amounts of information than volatile memory. Storage device 712 may further be configured for long-term storage of information. In some examples, storage devices 712 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 700, in some examples, also includes one or more communication modules 710. Computing device 700 in one example uses communication module 710 to communicate with external devices via one or more networks, such as one or more wireless networks. Communication module 710 may be a network interface card, such as an Ethernet card, an optical transceiver, a radio frequency transceiver, or any other type of device that can send and/or receive information. Other examples of such network interfaces include Bluetooth, 3G, 4G, LTE, 5G, Wi-Fi radios, and Near-Field Communications (NFC), and Universal Serial Bus (USB). In some examples, computing device 700 uses communication module 710 to wirelessly communicate with an external device such as via public network such as the Internet. Computing device 700 also includes, in one example, one or more input devices 706. Input device 706, in some examples, is configured to receive input from a user through tactile, audio, or video input. Examples of input device 706 include a touchscreen display, a mouse, a keyboard, a voice responsive system, video camera, microphone, or any other type of device for detecting input from a user.

One or more output devices 708 may also be included in computing device 700. Output device 708, in some examples, is configured to provide output to a user using tactile, audio, or video stimuli. Output device 708, in one example, includes a display, a sound card, a video graphics adapter card, or any other type of device for converting a signal into an appropriate form understandable to humans or machines. Additional examples of output device 708 include a speaker, a light-emitting diode (LED) display, a liquid crystal display (LCD), or any other type of device that can generate output to a user.

Computing device 700 may include operating system 716. Operating system 716, in some examples, controls the operation of components of computing device 700, and provides an interface from various applications such as breath intoxicant detection application 720 to components of computing device 700. For example, operating system 716, in one example, facilitates the communication of various applications such as breath intoxicant detection application 720 with processors 702, communication unit 710, storage device 712, input device 706, and output device 708. Applications such as breath intoxicant detection application 720 may include program instructions and/or data that are executable by computing device 700. As one example, breath intoxicant detection application 720 may include instructions that cause computing device 700 to perform one or more of the operations and actions described in the examples presented herein. Instead of a breath intoxicant detection application 720, the system may include an intoxication interlock application, a personal monitoring application, a substance detection application, or other applications.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

The claims:

1. A method of detecting THC compounds, comprising:
receiving an exhaled breath sample;
heating a capture structure to a temperature to vaporize THC compounds;
oxidizing the vaporized THC compounds on a detector element, wherein the detector element comprises a THC targeted enzyme substrate; and
detecting an amount of the THC compounds present in the exhaled breath sample.

2. The method of claim 1, wherein the detector element comprises an electrochemical sensor.

3. The method of claim 2, wherein the electrochemical sensor comprises a fuel cell, a chemiresistor, or a voltametric sensor.

4. The method of claim 1, wherein the vaporized THC compounds are oxidized on the detector element by contacting the THC targeted enzyme substrate.

5. The method of claim 4, wherein the vaporized THC compounds undergo an enzymatic oxidation reaction upon contacting the THC targeted enzyme substrate.

6. The method of claim 5, wherein an electric current is generated by the enzymatic oxidation reaction.

7. The method of claim 6, wherein the electric current generated is measured by the detector element.

8. The method of claim 7, wherein the electric current generated is proportional to the amount of the THC compounds present in the exhaled breath sample.

9. The method of claim 5, wherein the enzymatic oxidation reaction generates a secondary reaction product.

10. The method of claim 9, wherein the secondary reaction product is hydrogen peroxide.

11. The method of claim 9, wherein an amount of the secondary reaction product is measured by the detector element.

12. The method of claim 11, wherein the amount of the secondary reaction product is proportional to the amount of the THC compounds present in the exhaled breath sample.

13. The method of claim 1, wherein the vaporized THC compounds are reduced on the detector element by contacting the THC targeted enzyme substrate.

14. The method of claim 13, wherein the vaporized THC compounds undergo a THC reduction reaction upon contacting the THC targeted enzyme substrate.

15. The method of claim 14, wherein an electric current is generated by the THC reduction reaction, and wherein the electric current generated is measured by the detector element.

16. The method of claim 15, wherein the electric current generated is proportional to the amount of the THC compounds present in the exhaled breath sample.

17. The method of claim 1, wherein the capture structure receives the exhaled breath sample.

18. The method of claim 1, wherein the capture structure is heated to a temperature above 157° C.

19. A detection device comprising:
a capture structure for receiving a component of an exhaled breath sample;
a heating element configured to vaporize the component of the exhaled breath sample;
a detector element comprising a targeted enzyme substrate;
wherein the detector element is configured to detect an amount of the component.

20. The detection device of claim 19, wherein the component comprises cannabis compounds, glucose, ethanol, acetone, carbon monoxide, nitric oxide, ethane, isoprene, pentane, and water.
